(19) United States
(12) Patent Application Publication
(10) Pub. No.: US 2025/0366903 A1
(43) Pub. Date: Dec. 4, 2025

(54) DEVICES AND METHODS FOR LEAFLET CUTTING

(71) Applicant: Evalve, Inc., Santa Clara, CA (US)

(72) Inventors: Richard Thomas Childs, San Francisco, CA (US); Scott C. Mosher, San Francisco, CA (US); Lauren Troxler Harvey, Austin, TX (US); Koji J. Kizuka, Redwood City, CA (US); Ajitkumar B. Nair, Pleasanton, CA (US); Laura M. Kalvass, Mountain View, CA (US)

(21) Appl. No.: 19/306,640

(22) Filed: Aug. 21, 2025

Related U.S. Application Data
(63) Continuation of application No. 17/306,398, filed on May 3, 2021, now Pat. No. 12,414,811.
(60) Provisional application No. 63/020,669, filed on May 6, 2020.

(51) Int. Cl. A61B 18/14; A61B 18/00

(52) U.S. Cl. CPC A61B 18/1442; A61B 18/1492; A61B 2018/00369; A61B 2018/0072; A61B 2018/1452

(57) ABSTRACT

A system for cutting leaflet tissue at a cardiac valve may comprise a guide catheter having a proximal end and a distal end, wherein the distal end of the guide catheter is steerable to a position above a cardiac valve. The system may also include a handle coupled to the proximal end of the guide catheter, the handle comprising at least one control configured to steer the guide catheter to the position above the cardiac valve. Finally, the system may comprise a cutting mechanism routable through the guide catheter and able to be positioned at the distal end of the guide catheter, the cutting mechanism configured to cut a portion of leaflet tissue of the cardiac valve.

### CROSS-REFERENCE TO RELATED APPLICATIONS

This application claims priority to U.S. patent application Ser. No. 17/306,398, filed May 3, 2021, which claims priority to U.S. Provisional Patent Application No. 63/020,669, filed May 6, 2020, the entire contents of which are incorporated by reference herein.

### BACKGROUND OF THE INVENTION

The mitral valve controls blood flow from the left atrium to the left ventricle of the heart, preventing blood from flowing backwards from the left ventricle into the left atrium so that it is instead forced through the aortic valve for delivery of oxygenated blood throughout the body. A properly functioning mitral valve opens and closes to enable blood flow in one direction. However, in some circumstances the mitral valve is unable to close properly, allowing blood to regurgitate back into the atrium.

Mitral valve regurgitation has several causes. Functional mitral valve regurgitation is characterized by structurally normal mitral valve leaflets that are nevertheless unable to properly coapt with one another to close properly due to other structural deformations of surrounding heart structures. Other causes of mitral valve regurgitation are related to defects of the mitral valve leaflets, mitral valve annulus, or other mitral valve tissues.

The most common treatments for mitral valve regurgitation rely on valve replacement or repair including leaflet and annulus remodeling, the latter generally referred to as valve annuloplasty. One technique for mitral valve repair which relies on suturing adjacent segments of the opposed valve leaflets together is referred to as the “bowtie” or “edge-to-edge” technique. While these techniques can be effective, they usually rely on open heart surgery where the patient's chest is opened, typically via a sternotomy, and the patient is placed on cardiopulmonary bypass. The need to both, open the chest, and place the patient on bypass, is traumatic and has an associated high mortality and morbidity rate. In some patients, a fixation device can be installed into the heart using minimally invasive techniques. The fixation device can hold the adjacent segments of the opposed valve leaflets together and may reduce mitral valve regurgitation. One such device used to clip the anterior and posterior leaflets of the mitral valve together is the MitraClip® fixation device, sold by Abbott Vascular, Santa Clara, California, USA.

However, sometimes after a fixation device is installed, undesirable mitral valve regurgitation can still exist, or can arise again. For patients requiring re-intervention, the presence of a fixation device in their mitral valves obstructs transcatheter mitral valve replacement. These patients may also be considered too frail to tolerate open-heart surgery, so they are left with no viable options to further improve the function of their mitral valve.

Accordingly, it would be desirable to provide alternative and additional methods, devices, and systems for removing or disabling fixation devices that are already installed in preparation for the installation of an artificial, replacement mitral valve. The methods, devices, and systems may be useful for repair of tissues in the body other than heart valves. At least some of these objectives will be met by the invention described hereinbelow.

### BRIEF SUMMARY OF THE INVENTION

Implementations of the present invention solve one or more problems in the art with systems, methods, and apparatus configured to cut leaflet tissue at a cardiac valve. The system may comprise a guide catheter having a proximal end and a distal end, wherein the distal end of the guide catheter is steerable to a position above a cardiac valve. The system may also include a handle coupled to the proximal end of the guide catheter, the handle comprising at least one control configured to steer the guide catheter to the position above the cardiac valve. Finally, the system may comprise a cutting mechanism, routable through the guide catheter and able to be positioned at the distal end of the guide catheter, configured to cut a portion of leaflet tissue of the cardiac valve.

A method for cutting leaflet tissue at a cardiac valve within a body may comprise positioning a guide catheter, having a proximal and a distal end such that the distal end of the guide catheter is positioned at a cardiac valve. The method may further comprise routing a cutting mechanism through the guide catheter such that the cutting mechanism extends to the distal end of the guide catheter, wherein the cardiac valve is associated with an interventional implant that approximates adjacent leaflets of the cardiac valve, and a cutting mechanism extends from the guide catheter. Also, the method may include positioning the hook catheter to place the cutting mechanism into contact with leaflet tissue located adjacent to the interventional implant and actuating the cutting mechanism to cut at a portion of least one leaflet of the approximated adjacent leaflet.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an embodiment of a delivery system that may be utilized for guiding and/or delivering a cutting mechanism to a cardiac valve;

FIG. 2 is a schematic representation of a human heart with an interventional implant affixed between the anterior and posterior leaflets of the mitral valve and a guiding catheter extending from a transseptal puncture and positioned above the mitral valve;

FIGS. 3A-3D illustrate an embodiment of a cutting mechanism according to the present disclosure shown in use in association with a cardiac valve;

FIGS. 4A-4F illustrate an alternative embodiment of a cutting mechanism according to the present disclosure;

FIGS. 5A-5J illustrate an alternative embodiment of a cutting mechanism according to the present disclosure shown in use in association with a cardiac valve;

FIGS. 6A-6F illustrate an alternative embodiment of a cutting mechanism according to the present disclosure shown in use in association with a cardiac valve;

FIGS. 7A-7D illustrate alternative embodiment of a cutting mechanism according to the present disclosure shown in use in association with a cardiac valve; and

FIGS. 8A-8C illustrate yet another embodiment of a cutting mechanism according to the present disclosure shown in use in association with a cardiac valve.

### DETAILED DESCRIPTION OF THE PREFERRED EMBODIMENTS

Implementations of the present invention solve one or more problems in the art with systems, methods, and apparatus configured to cut leaflet tissue at a cardiac valve. More specifically, at least one embodiment of the present invention, may comprise a guide catheter having a proximal end and a distal end, wherein the distal end of the guide catheter is steerable to a position above a cardiac valve. The system may also include a handle coupled to the proximal end of the guide catheter, the handle comprising at least one control configured to steer the guide catheter to the position above the cardiac valve. Finally, the system may comprise a cutting mechanism routable through the guide catheter and able to be positioned at the distal end of the guide catheter, the cutting mechanism configured to cut a portion of leaflet tissue of the cardiac valve.

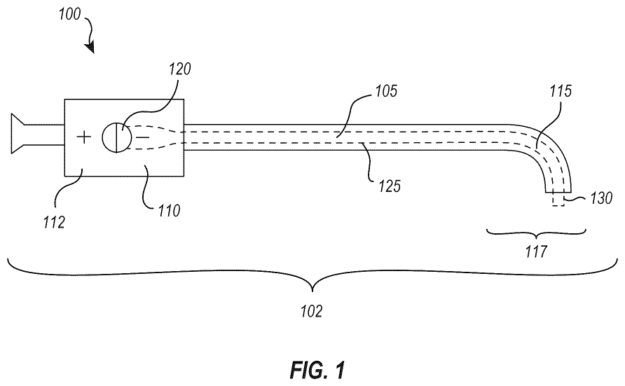

FIG. 1 illustrates an embodiment of a delivery system 100 that may be utilized for guiding and/or delivering a cutting mechanism to the cardiac valve. In at least one embodiment, the delivery system 100 includes a leaflet cutting system 100, which can include a delivery system 102 that may be utilized for guiding and/or delivering a cutting mechanism to the cardiac valve. The delivery system 102 can include a guide catheter 105 having a proximal end 112 and a distal end 115. The delivery system may comprise a handle 110 positioned on the proximal end 112 of the guide catheter 105. The guide catheter 105 may be operatively coupled to the handle 110. The guide catheter 105 may include a steerable portion 117 near the distal end 115 that can be steerable to enable the guiding and orienting of the guide catheter 105 through a patient's vasculature to a targeted treatment site, such as a mitral valve. For example, the handle 110 may include at least one control 120 (e.g., a dial, a switch, a slider, a button, etc.) that can be actuated to control the movement and curvature of a steerable portion 117 of the guide catheter 105.

In at least one embodiment, the at least one control 120 can be operatively coupled to one or more control lines 125 (e.g., pull wires) extending from the handle 110 through the guide catheter 105 to the distal end 115 of the guide catheter (e.g., through one or more lumens in the guide catheter 105). Actuation of the at least one control 120 may adjust the tensioning of one or more control lines 125 to steer the guide catheter 105 in a desired curvature and/or direction. FIG. 1 shows the handle 110 as having a single control 120 for providing steerability. Alternatively, a handle 110 may comprise more than one control 120 associated with any number of control lines.

While control lines or wires are described at various points in this application, it should be understood that references made throughout this application to control lines or wires may be a single wire or plurality of wires including or made of steel, titanium alloy, aluminum alloy, nickel alloy, other metals, a shape memory material (such as a shape memory alloy or shape memory polymer), inorganic polymer, organic polymer, ceramic, carbon materials, or other flexible material with sufficient tensile strength. For example, a control line 125 may be a steel cable. In another example, a control line 125 may be a monofilament suture. In another example, a control line 125 may be a multifilament suture. In yet another example, a control line 125 may be a braided suture.

It is desirable for guide catheter 105 to provide an adjustable distal end 115, which is capable of being positioned within a target body cavity in a desired orientation. Guide catheter 105 should have a large lumen diameter to accommodate the passage of a variety of devices, such as the various embodiments of the cutting mechanisms discussed hereinafter, and should have good wall strength to avoid kinking or collapse when bent around tight curves, and should have good column, tensile, and torsional strength to avoid deformation when the devices are passed through the lumen and torqued or translated. Guide catheter 105 should provide for a high degree of controlled deflection at its distal end 115, but should not take up significant lumen area to allow for passage of interventional devices, such as the cutting mechanisms discussed below. Further, guide catheter 105 should be able to be positioned in a manner which allows compound curves to be formed, for example curvature within more than one plane. Such manipulation should also allow fine control over distal end 115 to accommodate anatomical variations within the same type of body cavity and for use in different types of body cavities.

The guide catheter 105 may comprise a main body made of or including a flexible material. The main body may be made of or include a variety of flexible materials, such as thermoplastic elastomers (TPE). In some embodiments, the main body may be a polyether block amide (PEBA or PEBAX). The main body may have a constant durometer or may have varying durometer that varies along its longitudinal length or that varies in different portions of the body. For example, the main body of guide catheter 105 may be made of or include a body material having a durometer of 25D to 75D. In another example, the main body of guide catheter 105 may be made of or include a body material that has a durometer of about 45 D. In at least one embodiment, the body material may include PEBAX 4533. In at least another embodiment, the body material may include PEBAX 3533.

The guide catheter 105 preferably defines a central lumen, extending axially through its entire length, through which other elongate elements, such as the cutting mechanisms may be inserted for accessing a treatment site. The central lumen may also include a central lumen lining on an inner surface thereof. In some embodiments, the central lumen lining may be a protective material that protects the interior walls from damage due to another element of the elongated member moving through or within the central lumen. In other embodiments, the central lumen lining may include a lubricious coating that reduces friction between the interior wall and another element of the elongated member moving through or within the central lumen. The central lumen lining may include PEBA, polytetrafluoroethylene (“PTFE”), polyetheretherketone (“PEEK”), other polymers, thermoplastic polyurethane (“TPU”), polyethylene with pebble stone surface, silicone oil stainless steel, Nitinol, other metals, or combinations thereof. In at least one embodiment, the central lumen lining may include a plurality of PEBA materials having different durometers.

In other embodiments, the guide catheter 105 may also have an outer layer. In some embodiments, the outer layer may be made of or include a single material or may be made of or include different materials to impart different handling characteristics to the guide catheter 105. For example, the outer layer may be made of or include softer materials to promote flexibility of the guide catheter 105. In other examples, the outer layer may be made of or include stiffer materials to promote pushability and/or torqueability of the guide catheter 105. In yet other examples, the outer layer may include lubricious materials to reduce friction between the guide catheter 105 and the body lumen of the patient. The outer layer may include PEBA, polytetrafluoroethylene (“PTFE”), polyetheretherketone (“PEEK”), other polymers, thermoplastic polyurethane (“TPU”), polyethylene with pebble stone surface, silicone oil stainless steel, Nitinol, other metals, or combinations thereof. In at least one embodiment, the outer layer may include a plurality of PEBA materials having different durometers.

In some embodiments, the outer layer of guide catheter 105 may also include a radiopaque marker to improve visualization of guide catheter 105 during a medical procedure. For example, the outer layer may include a barium sulfate (BaSO4), gold, platinum, platinum iridium, iodine, other radiopaque materials, or combinations thereof on a distal portion of guide catheter 105. In at least one embodiment, one or more additional radiopaque markers may be longitudinally located at one or more intermediate locations along the length of guide catheter 105.

The curves of guide catheter 105 may be formed by any suitable means. In some embodiments, one or more of the curves are preset so that the curve is formed by shape memory. For example, guide catheter 105 may be comprised of a flexible polymer material in which a curve is preset by heating. When guide catheter 105 is loaded on a guidewire, dilator, obturator or introductory device, the flexibility of guide catheter 105 can allow it to follow the shape or path of the introductory device for proper positioning within the body. When the introductory device is pulled back and/or removed, guide catheter 105 can then resume the shape memory configuration which was preset into the catheter.

Alternatively, the curves may be formed or enhanced with the use of one or more steering mechanisms. In some embodiments, the steering mechanism comprises at least one control wire or pull wire attached to one of the guide catheter 105, wherein actuation of the steering mechanism applies tension to the at least one pull wire whereby the curve is formed. The pull wires can extend through the central lumen or through individual lumens in the wall of guide catheter 105. It may be appreciated that more than one pull wire may extend through any given lumen. The presence of each pull wire allows curvature of guide catheter 105 in the direction of the pull wire. For example, when pulling or applying tension to a pull wire extending along one side of the catheter, the catheter will bend, arc or form a curvature toward that side. To then straighten the catheter, the tension may be relieved for recoiling effects or tension may be applied to a pull wire extending along the opposite side of the catheter. Therefore, pull wires are often symmetrically placed along the sides of the catheter.

Thus, in some embodiments at least two pull wires are attached in diametrically opposed locations wherein applying tension to one of the pull wires curves the catheter in one direction and applying tension to the pull wire attached in the diametrically opposed location curves the catheter in another direction opposite to the one direction. The diametrically opposed pull wires may be considered a set. Any number of sets may be present in a catheter to provide unlimited directions of curvature. In some embodiments, the steering mechanism can comprise at least four pull wires wherein two of the at least four pull wires are attached to the guide catheter in diametrically opposed locations and another two of the at least four pull wires are attached to the guide catheter in diametrically opposed locations. In other words, the catheter may include two sets of pull wires, each set functioning in an opposing manner as described. When the two sets of pull wires are positioned so that each pull wire is 90 degrees apart, the catheter may be curved so that the distal end is directed from side to side and up and down. In other embodiments, the steering mechanism comprises at least three pull wires, each pull wire symmetrically positioned approximately 120 degrees apart. When tension is applied to any of the pull wires individually, the catheter is curved in the direction of the pull wire under tension. When tension is applied to two pull wires simultaneously, the catheter is curved in a direction between the pull wires under tension. Additional directions may also be achieved by various levels of tension on the pull wires. It may be appreciated that any number, combination and arrangement of pull wires may be used to direct the catheters in any desired direction.

In some embodiments, a portion of one of guide catheter 105 can comprise one or more articulating members. In this case, the at least one pull wire is attached to one of the articulating members so that the curve is formed by at least some of the articulating members. Each pull wire is attached to the catheter at a location chosen to result in a particular desired curvature of the catheter when tension is applied to the pull wire. For example, if a pull wire is attached to the most distal articulating member in the series, applying tension to the pull wire will compress the articulating members proximal to the attachment point along the path of the pull wire. This results in a curvature forming in the direction of the pull wire proximal to the attachment point. It may be appreciated that the pull wires may be attached to any location along the catheter and is not limited to attachment to articulating members. Typically, the articulating members comprise inter-fitting domed rings but may have any suitable shape.

It may also be appreciated that curves in guide catheter 105 may be formed by any combination of mechanisms. For example, a portion of guide catheter could form a curve by shape memory while a different portion of guide catheter could form a curve by actuation of a steering mechanism.

The steering mechanisms may be actuated by manipulation of actuators located on handle 110. The handle 110 can be connected with the proximal end of the guide catheter 105 and remains outside of the body. One or more actuators or controls 120 can be provided on handle 110 and may have any suitable form, including buttons, levers, knobs, switches, toggles, dials, or thumbwheels, to name a few. When pull wires are used, each actuator may apply tension to an individual pull wire or to a set of pull wires. The handle may also include one or more locking mechanisms configured to interface with, and selectively lock into place, one or more of the controls 120.

In at least one embodiment, the handle 110 includes at least one control 120 for actuating and/or adjusting one or more components of a cutting mechanism 130. As shown in FIG. 1, the cutting mechanism 130 is configured to extend beyond the distal end 115 of the guide catheter 105. In at least one embodiment, the cutting mechanism 130 is routable through the guide catheter 105 and retractable into the guide catheter 105. The at least one control 120 may control the cutting mechanism's 130 extension from, and retraction into, the guide catheter 105. Additionally or alternatively, the at least one control 120 may be configured to provide selective actuation of the cutting mechanism 130. The at least one control 120 may be operatively connected to one or more additional elements of the cutting mechanism 130. The cutting mechanism 130 is shown here in generic form as a dashed line, and therefore represents any of the cutting mechanism 130 embodiments described herein.

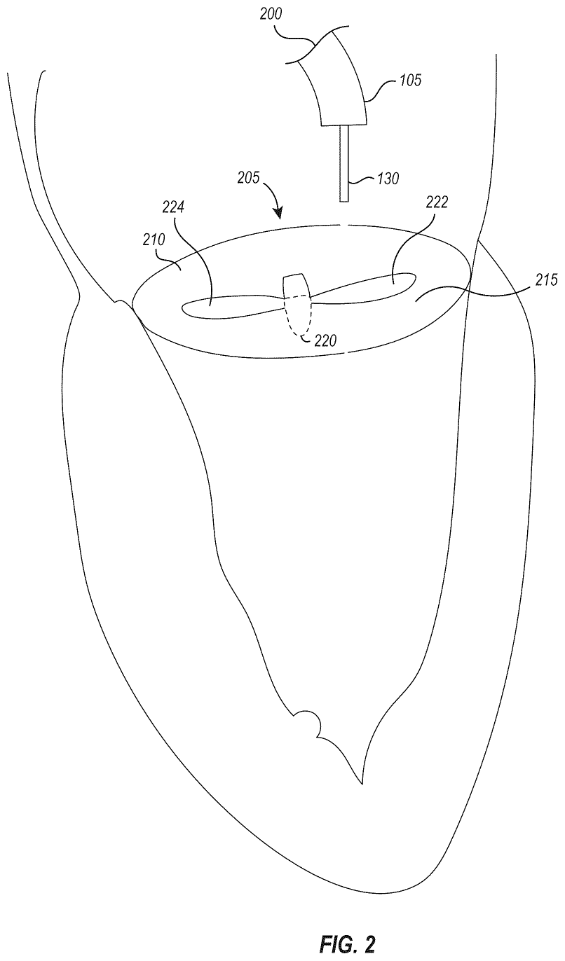

FIG. 2 is a schematic representation of a human heart with an interventional implant affixed between the anterior and posterior leaflets of the mitral valve. The mitral valve comprises a posterior mitral leaflet 210 and an anterior mitral leaflet 215. FIG. 2 also shows an interventional implant (e.g., MitraClip®) 220, which has previously been affixed to the leaflets in an effort to reduce regurgitation by approximating the adjacent leaflets 210 and 215. As shown, the affixation of implant 220 to the leaflets creates a first orifice 222 and a second orifice 224 located on opposing sides of implant 220 and between the anterior mitral leaflet 215 and the posterior mitral leaflet 210. And, as discussed above, if further treatment is required in the form of the installation of an artificial, replacement mitral valve, the prior clip implant 220 must be detached from one or both of the leaflets before the replacement valve can be implanted.

In use, the leaflet cutting system 100 can be inserted into, and navigated through a patient's vasculature in a conventional manner to arrive in the patient's heart. As also illustrated in FIG. 2, a distal end portion of the leaflet cutting system 100 can be inserted through the interatrial septum 200 of the heart and positioned above the mitral valve in preparation for a leaflet cutting procedure. In at least one embodiment, the distal end 115 of the guide catheter 105 can include one or more radiopaque or echogenic markers to aid in accurate positioning. One skilled in the art will appreciate that the positioning of the distal end portion of the guide catheter 105 of the leaflet cutting system 100 in FIG. 2 is merely exemplary, and the present disclosure is not limited to the specific positioning shown.

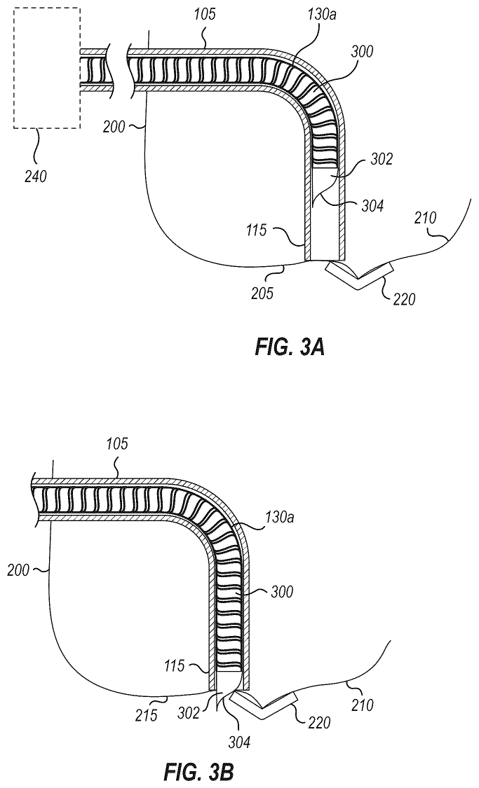

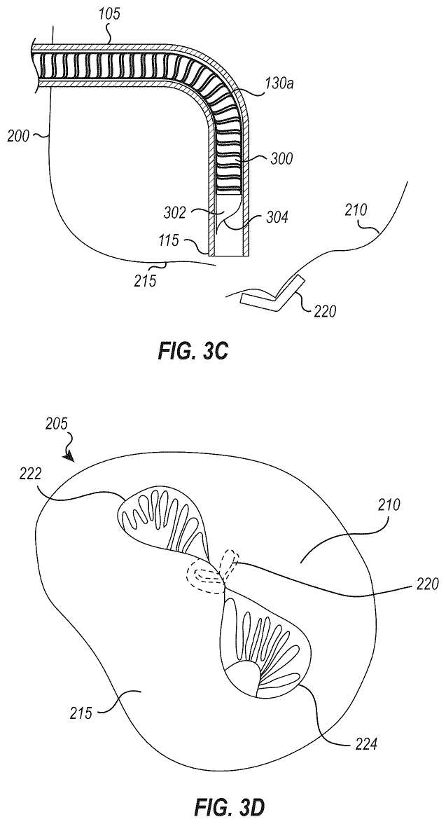

Referring next to FIGS. 3A-3D, which illustrate a first embodiment of a leaflet cutting system 100. System 100 includes components corresponding to those described above in connection with FIG. 1, which are designated by the same reference numerals throughout. As shown in FIGS. 3A-3D, system 100 also includes a cutting mechanism 130a positioned within, and routed through, the guide catheter 105. As shown, the cutting mechanism 130a may comprise an elongate inner catheter or hypotube 300, extending from a proximal end (not shown in FIG. 3) to a distal end 302 of the leaflet cutting system 100. The proximal end of the cutting mechanism 130a can be operatively coupled to the handle 110, and handle 110 provided with controls adapted to manipulate the cutting catheter 130a, including advancing, retracting and/or rotating the cutting mechanism 130a relative to the guide catheter 105.

As shown in FIG. 3A, cutting mechanism 130a is positioned within the interior of guide catheter 105 during advancement and positioning of the system 100 above the mitral valve 205. As discussed below, once the distal end 115 of guide catheter 105 is properly positioned above the mitral valve 205 and in proper alignment with interventional implant 220, the handle 110 can be manipulated to cause cutting mechanism 130a to advance relative to guide catheter 105 and to pass through the tissue of one of the leaflets 210, 215, thereby cutting the affected leaflet and separating the interventional implant 220 from the affected leaflet.

In this embodiment, the cutting mechanism 130a can have at its distal end 302, a sharped end 304 that terminates in a point. The sharpened end 304 may have a circular or oval cross-sectional shape. The sharpened end 304 can also have tapered, sharpened edges or blades adjacent to and extending from the point, which are adapted to slide through the leaflet tissue located between the first and second orifices 222, 224 and adjacent the interventional implant 220. In this and other embodiments described herein, the components of cutting mechanism 130a may be formed from the same or different materials, including but not limited to stainless steel or other metals, Elgiloy®, nitinol, titanium, tantalum, metal alloys or polymers.

FIG. 3B also shows how the hypotube 300 may be advanced to cause the sharpened end 304 to extend from the distal end 115 of the guide catheter 105 and cut a portion of the anterior mitral leaflet 215. The point of sharpened end 304 helps anchor and stabilize the leaflet tissue as the sharpened, cutting edges of the cutting mechanism 130a are advanced through and cut the leaflet tissue. The circular or oval cross section of sharpened end 304 of the cutting mechanism 130a cause the sharpened end to cut through the leaflet tissue in an arc around the interventional implant and extending from the first orifice 222 to the second orifice 224, as schematically illustrated in FIG. 3D. After the sharpened end 304 of cutting mechanism 130a advances and cuts through the tissue of the anterior mitral leaflet 215, the interventional implant 220 may remain attached to the posterior mitral leaflet 210, as shown in FIG. 3C, thereby reducing the risk that the interventional implant 220 will interfere with functioning of the left ventricular outflow tract. Additionally or alternatively, the posterior mitral leaflet 210 may also be cut in a similar manner with little or acceptable risk of left ventricular outflow tract interference. In at least one embodiment, the interventional implant 220 is removed from the patient.

FIG. 3D is a top perspective view of a mitral valve showing the cut portion of the anterior mitral leaflet 215. As shown, the sharpened end 304 may cut an arc in the leaflet 210 around the interventional implant 220. The hypotube 300 may be sized depending on the number of interventional implants 220 that are located within the cardiac valve. Additionally or alternatively, a single hypotube 300 may be used to cut multiple interventional implants 220 within a cardiac valve.

In this and other embodiments described herein, inner catheter or hypotube 300 should preferably have sufficient flexibility as to be able to conform to bends formed by guide catheter 105. Additional flexibility to accommodate bending may be provided in certain regions of hypotube 300 by a series of laser cuts formed in the outer wall of hypotube 300. In addition, hypotube 300 should also provide sufficient compressive strength to permit forces to be transmitted through hypotube 300, from the proximal end to the distal end, sufficient to cause the sharpened end 304 of hypotube 300 to cut through the leaflet tissue.

In an alternate embodiment, an indeflator 240 can be attached to the guide catheter 105 at or near its proximal end. The indeflator 240 can be configured to create a vacuum transmitted through the lumen of the guide catheter 105. When the distal end of the guide catheter is positioned against the leaflet, a negative pressure can be applied to the leaflet tissue, thereby stabilizing nearby leaflet tissue located adjacent the interventional implant 220. While leaflet tissue adjacent to the clip implant 220 is held in place by the negative vacuum pressure applied through the distal end of the guide catheter, the hypotube 300 can be advanced relative to the guide catheter 105, thereby causing the sharpened end 304 to pass through and cut the leaflet tissue surrounding the clip implant 220. In this alternate embodiment, a distal end portion of the guide catheter 105 may comprise various cross-sectional shapes including a U-shape. For example, the distal end 115 of the guide catheter 105 can comprise a flexible U-shape that may collapse when positioned within a delivery sheath and then expand when the guide catheter is advanced to extend beyond the distal end of the delivery sheath.

In another alternate embodiment, in addition to the sharpened edges located at the distal end of the cutting mechanism 130a used for mechanical cutting, the cutting mechanism 130a could also include an electrical conductor (not shown) that extends along its entire length, which conductor is electrically coupled at a distal end to the sharpened cutting end 304 and which is also electrically coupled at a proximal end to a source for selectively applying electrosurgical energy, such as an electrosurgical generator. In that case, the cutting of leaflet tissue could be achieved by mechanical cutting, by the application of electrosurgical energy, or a combination of both.

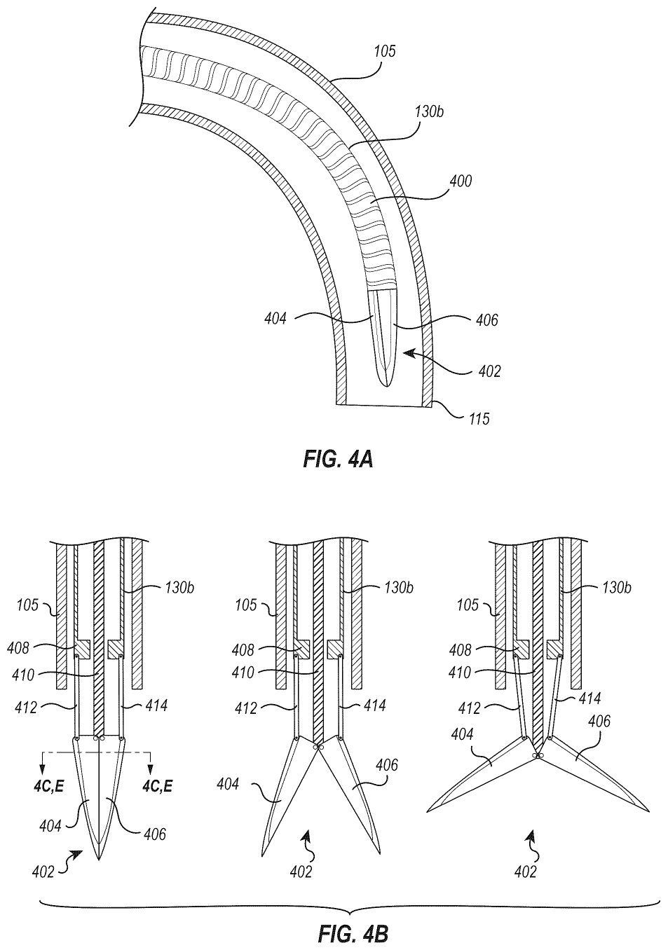

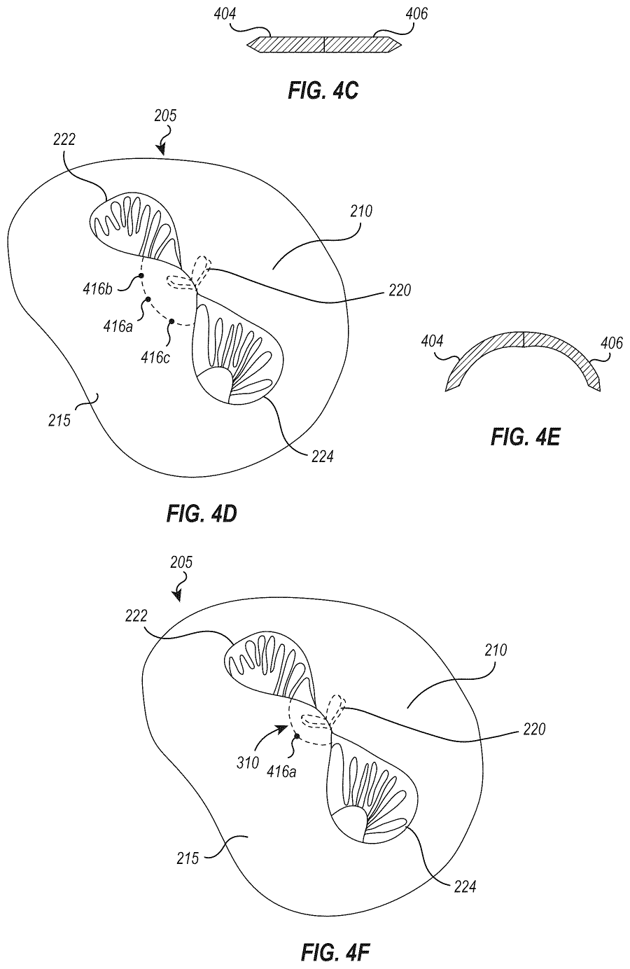

Reference is next made to FIGS. 4A-4F, which illustrate a second embodiment of a cutting mechanism 130b. This embodiment also includes components corresponding to those described above in connection with FIG. 1, which are designated by the same reference numerals throughout. FIG. 4A is a side sectional view of the cutting mechanism 130b shown within the distal end 115 of the guide catheter 105. As shown, the cutting mechanism 130b may comprise an inner catheter or hypotube 400 having a sharpened, cutting end 402, which terminates at its distal end in a sharpened point. This embodiment, the sharpened cutting end 402 can include two blades 404 and 406 joined at a pivot point. The two blades 404 and 406 may each comprise a cutting edge disposed on an outer edge of each blade as illustrated in FIG. 4B. The hypotube 400 may be configured to be selectively extended from the distal end 115 of the guide catheter 105, thereby allowing the two blades 404 and 406 to engage with a portion of leaflet tissue.

In this embodiment, the blades 404 and 406 can be connected to the hypotube 400 in such a way that, when actuated, the blades pivot outwardly away from one another in an arcuate path in a “reverse-scissor” fashion. In the illustrated embodiment, this can be accomplished as follows. A tip ring 408 can be integrally attached to the distal end of the hypotube. Tip ring 408 can include a center lumen that allows an actuating rod 410 to extend therethrough as shown in FIG. 4B. The actuating rod 410 can be pivotally connected at its distal end to a center portion of the proximal ends of each of blades 404 and 406 as shown. Links 412 and 414 can also be provided, which connect a distal end of tip ring 408 to an outer portion of the proximal ends of each of blades 404 and 406. While not explicitly shown in FIG. 4, hypotube 400 and actuating rod 410 can run the entire length of delivery system 102 and can be operatively coupled at their proximal ends to handle 110, and handle 110 can be provided with suitable controls to allow manipulation of hypotube 400 and actuating rod 410 relative to one another to cause blades 404 and 406 to open and close relative to one another. It will be appreciated that advancing actuating rod 410 relative to hypotube 400 will cause blades 404 and 406 to open in an arcuate path away from one another, and that withdrawing actuating rod 410 relative to hypotube 400 will cause blades 404 and 406 to close and move toward one another. Alternatively, actuating rod 410 could be held stationary and hypotube 400 moved relative to actuating rod 410 to open and close blades 404 and 406. Other structures and mechanisms known to those skilled in the art could be adapted and used for opening and closing blades 404 and 406 and are intended to be encompassed within the scope of the invention.

The cross-sectional shape of blades 404 and 406 can be selected to influence that shape of the path they travel as they cut through leaflet tissue. For example, in one embodiment illustrated in FIG. 4C, blades 404 and 406 can have a relatively straight or flat cross-sectional shape, which will cause the blades to cut through the leaflet tissue in a relatively straight path. Referring to FIG. 4D, the cut through the leaflet tissue can be made in multiple segments. For example, with blades 404 and 406 closed, the sharpened end 402 can be inserted at a first insertion point 416a into and through leaflet 210. The sharpened point and sharpened edges of sharpened end 402 assist with the ease of insertion and also help stabilize the tissue adjacent to the interventional implant 220. Once inserted through the tissue, blades 404 and 406 can be actuated by means of handle 110, acting through hypotube 400 and actuating rod 410, to cause the blades to open, thereby cutting through the leaflet tissue located to either side of insertion point 416a. The cutting mechanism can then be withdrawn in a proximal direction, repositioned, re-advanced in a distal direction through the tissue at a second insertion point 418a, re-actuated, and then at a third insertion point 418c, and so and so forth until the process completed to create a continuous cut through the affected leaflet tissue around the interventional implant 220 and extending from the first orifice 222 to the second orifice 224. Alternatively, the opening and closing of blades 404 and 406 can simply be reciprocated as the guide catheter 105 is used to drag the cutting blades through the leaflet tissue.

In an alternate embodiment, as illustrated in FIGS. 4E-4F, the two blades 404 and 406 can be shaped such that they cut the portion of leaflet tissue in an arcuate path. As best shown in FIG. 4E, blades 404 and 406 can also be made to have a cross-sectional shape that is circular or oval in shape. As with the above, the pointed end of sharpened end 402 is advanced through an entry point 416a into the leaflet tissue. However, when actuated, the shape of blades 404 and 406 cause them to travel in an actuate path through the leaflet tissue, as illustrated in FIG. 4F.

The cut portion in FIG. 4F is shown on the anterior leaflet 215, however, the present invention is not limited to the positioning of shape or cut. In at least one embodiment, the posterior mitral leaflet 210 can also be cut. Additionally or alternatively, the interventional implant 220 may be removed from the patient.

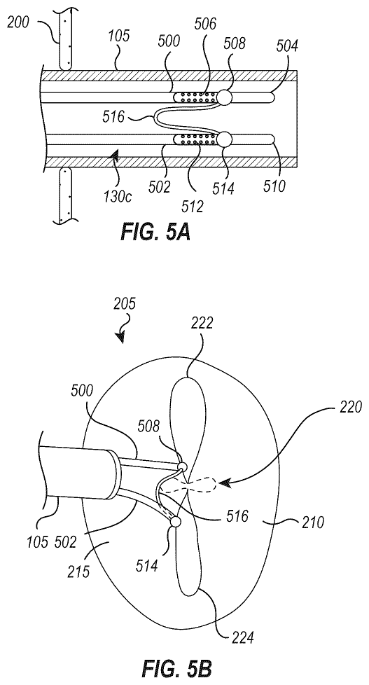

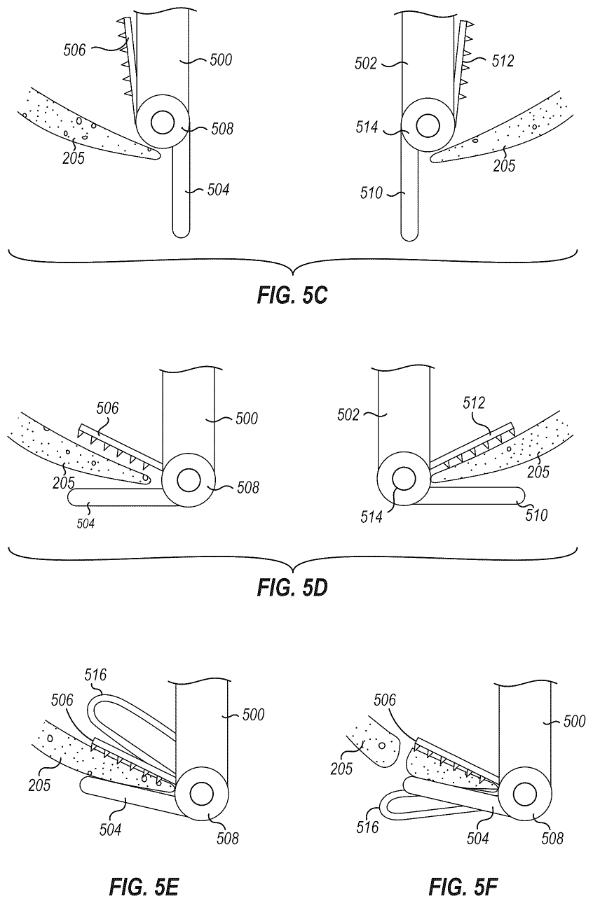

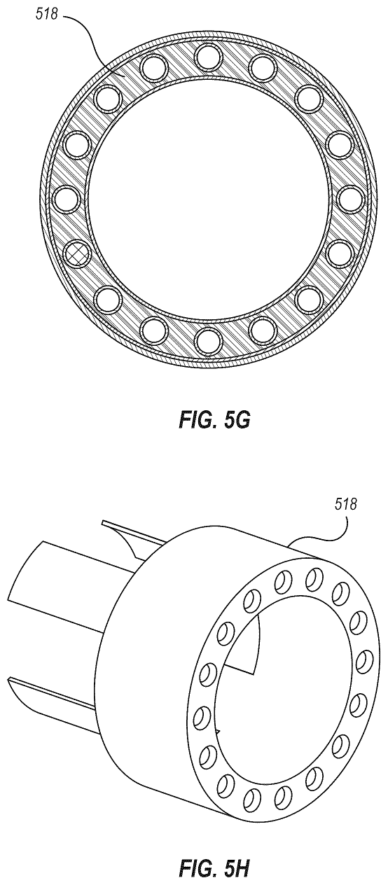

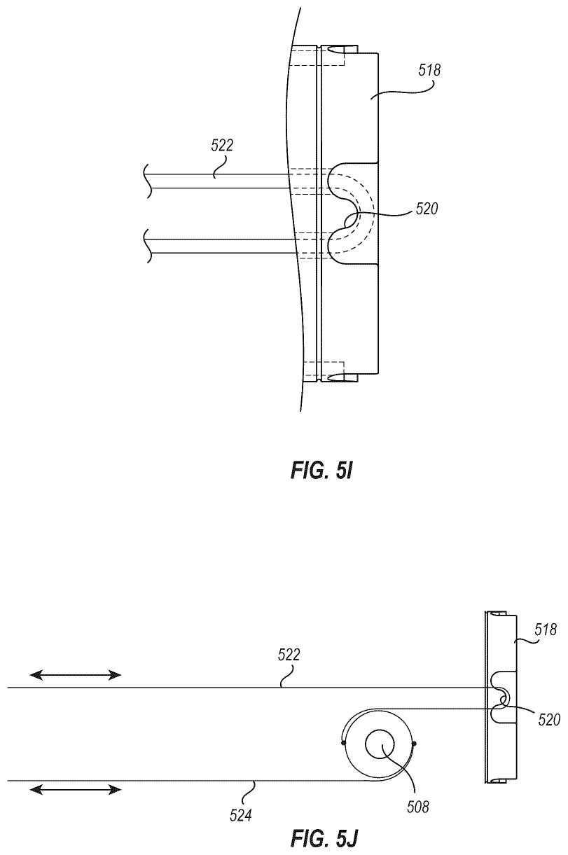

Reference is next made to FIGS. 5A-5J, which illustrate yet another embodiment of the cutting mechanism. In this embodiment, cutting mechanism 130c may comprise a first and a second delivery catheter 500 and 502, each comprising a proximal end and a distal end. The first delivery catheter 500 can include at its distal end a pair of grasping arms 504 and 506, which arms can be rotatably coupled to the distal end of delivery catheter 500 by a rotatable hub 508. Similarly, second delivery catheter 502 can include at its distal end a pair of grasping arms 510 and 512, which arms can be rotatably coupled to the distal end of delivery catheter 502 by a rotatable hub 514. One or more of grasping arms 504, 506, 510 and/or 512 may include one or more teeth to prevent slippage when the arms are engaged with leaflet tissue.

A cutting wire 516 may extend from the hub 508 of the first delivery catheter 500 to the 514 of the second delivery catheter 502. The cutting wire 516 may be configured to selectively provide electrosurgical energy to a secured portion of leaflet tissue, thereby cutting a portion of leaflet tissue when rotated around hubs 508 and 514. Additionally or alternatively, an adjustment in the tension of cutting wire 516 may cause the wire 516 to engage with and cut the secured leaflet tissue.

As shown in FIG. 5B, which is a top perspective view of a mitral valve, the first and second delivery catheters 500 and 502 may be configured to extend from the distal end 115 of the guide catheter 105. The interventional implant 220 creates a first orifice 222 and a second orifice 224 between the anterior mitral leaflet 215 and the posterior mitral leaflet 210 by approximating the adjacent leaflets 210 and 215. The first delivery catheter 500 may extend toward the first orifice 222 and thereby extend the first grasping arm 504 (not shown in FIG. 4B) of the first delivery catheter 500 into the first orifice 222 of the mitral valve. Similarly, the second delivery catheter 502 may extend toward the second orifice 224 and thereby extend the first grasping arm 510 (not shown in FIG. 4B) of the second delivery catheter 502 into the second orifice 224 of the mitral valve. The first grasping arms 504 and 510 may be configured to engage with leaflet tissue on the ventricular side of the mitral valve.

FIGS. 5C and 5D are a detailed side views of the distal end of delivery catheter pair 500 and 502 at various stages of positioning and deployment to attach and hold onto a portion of the tissue of one of the leaflets 205, with one delivery catheter 500 attaching to the leaflet 210 to one side of the interventional implant 220 and the other delivery catheter 502 attaching to the same leaflet 210 on the other side of the interventional implant 220. Once the guide catheter 105 is properly positioned above and aligned with the interventional implant 220, delivery catheters 500 and 502 are advanced relative to the guide catheter 105 to extend beyond the distal end 115 of guide catheter 105. The distal ends of delivery catheters 500 and 502 are advanced until grasping arms 504 and 510 pass through orifices 222 and 224 of the mitral valve on opposites side of the interventional implant 220, as shown in FIG. 5C. Guide catheter 105 can then be manipulated to reposition grasping arms 504 and 510 into direct contact with the edges of orifices 222 and 224, as also shown in FIG. 5C. Once that is done, grasping arms 504 and 506 of delivery catheter 500 can be rotated toward one another to clamp down on the leaflet tissue adjacent the first orifice 222 (and located to one side of the interventional device 220), and grasping arms 510 and 512 of delivery catheter 502 can similarly be rotated toward one another to clamp down on either side of the leaflet tissue adjacent the second orifice 224 (and located to the other side of the interventional device 220), as shown in FIG. 5D. Once the leaflet tissue is secured between grasping arms 504 and 506 and between grasping arms 510 and 512, then cutting wire 516 can be rotated to cut through the leaflet tissue and separate the interventional implant 220 from the affected leaflet, as illustrated in FIGS. 5E and 5F.

FIGS. 5G-5J illustrate one example of a mechanism of actuating the various components of cutting mechanism 130c described above. However, additional structures and mechanisms known to those skilled in the art could be adapted and used for controlling the selective, relative rotation of grasping arms 504, 506, 510 and 512, as well as that of cutting wire 516. In one embodiment, each of delivery catheters 500 and 502 can include at their respective distal ends a tip ring 518, having a plurality of lumens for accepting a plurality of control lines. Each control line, such as control line 522 shown in FIGS. 5H and 5I, can be coupled at the proximal end of the system 100 to a dedicated control located on handle 110. Separate control lines can be provided to separately and independently control selective movement of each of grasping arms 504, 506, 510 and 512, as well as movement of cutting wire 516. Certain control lines, such as control line 522, can extend the entire length of the system and pass around a saddle, such as saddle 520 formed in tip ring 518 between adjacent lumens, as shown in FIGS. 5I and 5J, so that the control line returns in a proximal direction for attachment to one of the rotatable hubs, such as rotatable hub 508, as shown in FIG. 5J. As further shown in FIG. 5J, control line 522 can be mechanically coupled to the periphery on one side of hub 508, and a second control line 524 can be mechanically coupled to the periphery of the other side of hub 508. As will be readily understood, selectively pulling on control line 522 will cause hub 508 to rotate in clockwise direction, and selectively pulling on control line 524 will cause hub 508 to rotate in counter-clockwise direction. By providing multi-part hubs and separate control lines for each component, relative movement and control of grasping arms 500 and 502, grasping arms 510 and 512, and cutting wire 516 can be effectuated from the proximal end of system 100 by means of one or more controls 120 provided on handle 110.

FIGS. 5C-5F illustrate how the grasping arms 504, 506, 510 and 512 are configured to secure a portion of the anterior mitral leaflet 215 therebetween and show the cutting wire 516 as cutting a portion of the anterior mitral leaflet 215. However, in at least one embodiment the posterior mitral leaflet 210 can be cut. The interventional implant 220 may additionally or alternatively be removed. As shown in FIGS. 5E and 5F, the cutting wire 516 may be rotated through the secured portion of the anterior mitral leaflet 215, thereby cutting the portion of leaflet tissue.

In at least one embodiment, the space between the first and second delivery catheters 500 and 502 may be adjusted based on number of interventional implants 220 within the mitral valve. Additionally or alternatively, the cutting mechanism 130c may be configured to retract into the distal end 115 of the guide catheter 105 after the portion of leaflet tissue is cut. One skilled in the art will appreciate that the positioning shown in FIGS. 5C-5F is merely exemplary and the present invention is not limited to the positioning shown.

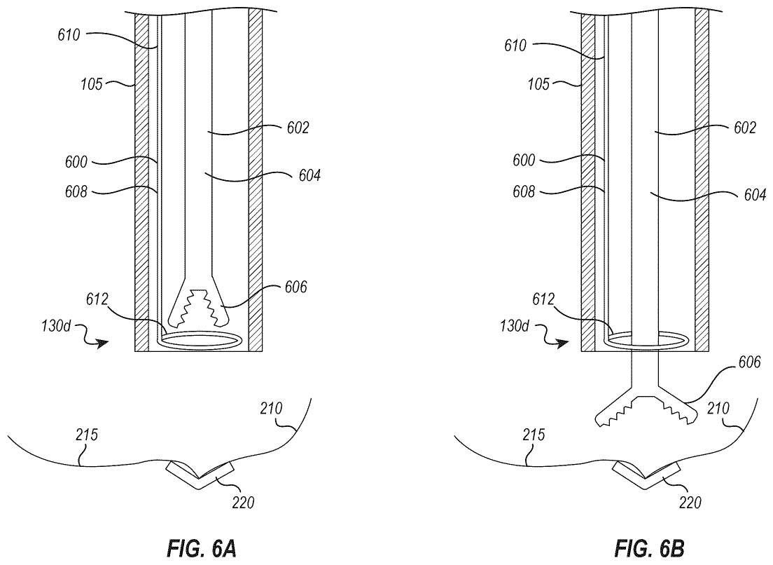

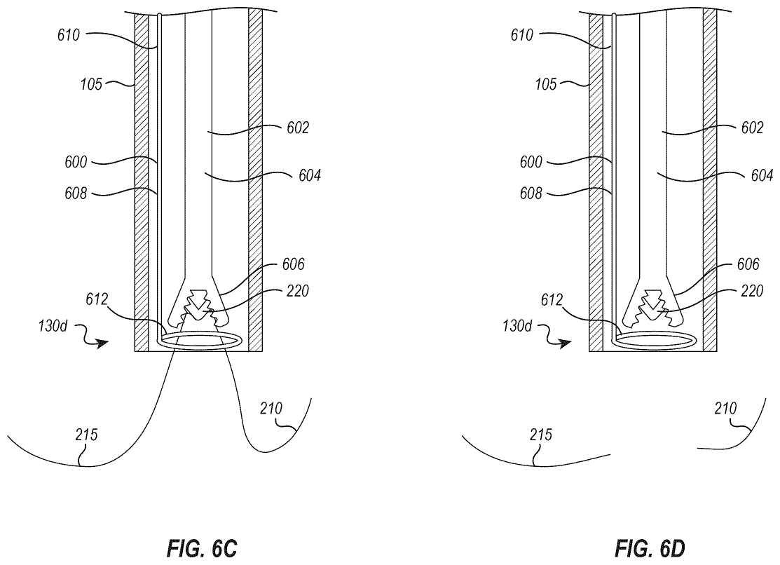

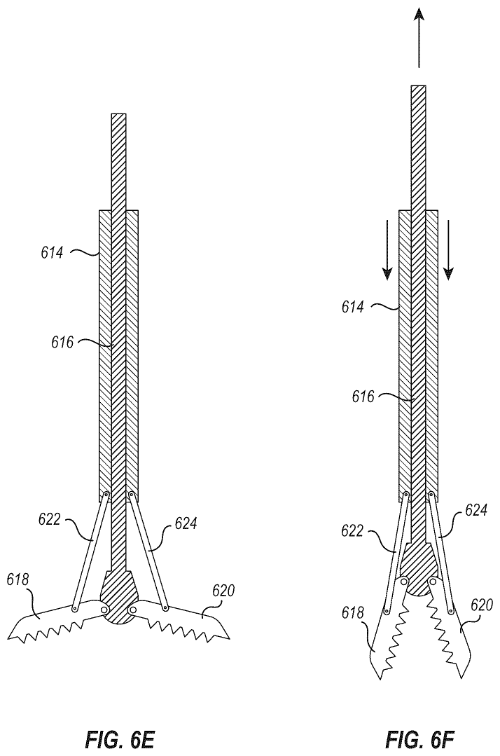

FIGS. 6A-6F are side sectional views and illustrate yet another alternative exemplary embodiment of a cutting mechanism shown in use in association with a cardiac valve, specifically a mitral valve. In this embodiment, the cutting mechanism 130d can include a clip grasping structure 602 and a cutting wire 608. FIG. 6B shows the distal end 115 of the guide catheter 105 positioned above the interventional implant 220, which approximates the anterior mitral leaflet 215 and posterior mitral leaflet 210. The cutting wire 608 can include an elongated portion 610 that terminates in a distal lasso or loop portion 612. The cutting wire 608 may extend from the handle 110 to the distal end 115 of the guide catheter 105. Further, the cutting wire 608 may be configured to selectively provide electrosurgical energy through the distal loop portion 612 to tissue in contact with distal loop portion 612. While the cutting wire 608 is shown as a separate component in FIGS. 6A-6D, a cutting electrode could also be integrally formed on the distal end 115 of guide catheter 105 and/or on the interior wall surface of guide catheter 105 adjacent the distal end 115.

FIGS. 6A-6F also illustrate a clip grasping structure 602 comprising an elongated portion 604 and a distal clamping portion 606, which may be routed through the guide catheter 105. As shown in FIG. 6B, the distal clamping portion 606 is extendable distally past the distal end 115 of the guide catheter 105. The distal clamping portion 606 may extend through the distal loop portion 612 of the cutting wire 608 when extending distally past the distal end 115 of the guide catheter 105.

The distal clamping portion 606 may be configured to secure an interventional implant 220, as shown in FIG. 6C. The distal clamping portion 606 may also be retractable proximally into the distal end 115 of the guide catheter 105 with the secured interventional implant 220. In at least one embodiment, the distal clamping portion 606 extends through the distal loop portion 612 of the cutting wire 608 when retracting proximally into the distal end 115 of the guide catheter 105 with the secured interventional implant 220. As shown in FIG. 6D, the distal loop portion 612 of the cutting wire 608 may be configured to cut a portion of leaflet tissue when the distal clamping portion 606 of the clip grasping structure 602 retracts proximally into the distal end 115 of the guide catheter 105 with the secured interventional implant 220. The distal loop portion 612 of the cutting wire 608 may also be selectively constricted to engage with and cut the portion of leaflet tissue. In at least one embodiment, the cutting wire 608 is configured to detach the interventional implant 220 from surrounding leaflet tissue.

FIGS. 6E and 6F illustrate one example of a mechanism of actuating the distal clamping portion 606 of grasping structure 602. However, additional structures and mechanisms known to those skilled in the art could be adapted and used for selectively opening, closing and clamping distal clamping portion 606. In this embodiment, grasping mechanism 602 can include an elongate hypotube 614, an actuating rod 606, a pair of grasping arms 618 and 620, and a pair of links 622 and 624. A proximal end of each grasping arm 618 and 620 can be pivotally coupled to the distal end of actuating rod 616. Links 622 and 624 can each be pivotally connected at one end to the distal end of hypotube 614 and can be pivotally connected at the other end to an intermediate location on grasping arms 618 and 620, respectively. It should be further understood that hypotube 614 and actuating rod 616 can extend the entire length of the system and can be operatively coupled at their proximal ends to suitable controls 120 located on handle 110 to control movement of hypotube 614 and actuating rod 616 relative to one another and, thus, causing grasping arms to open and close. The tissue engaging surface of grasping arms 618 and 620 can also include one or more teeth to engage leaflet tissue adjacent the interventional implant and prevent slippage.

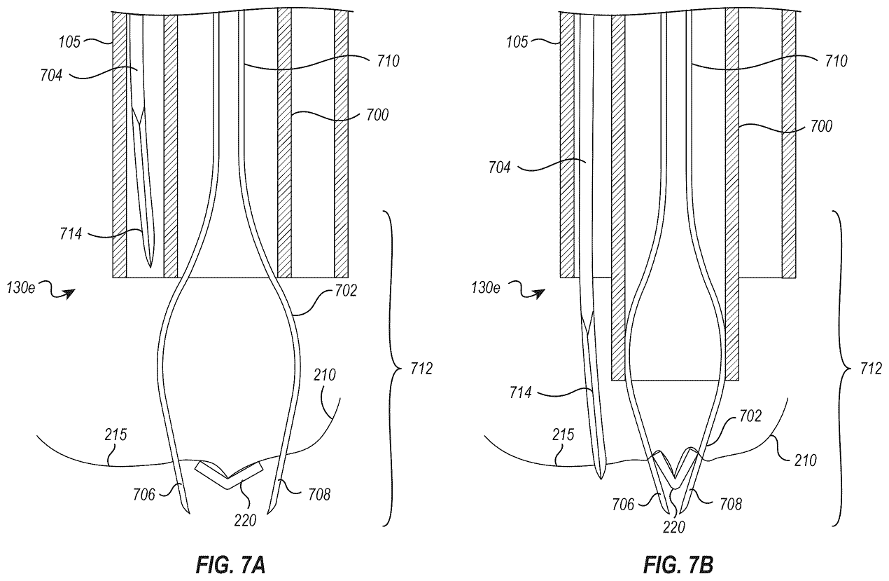

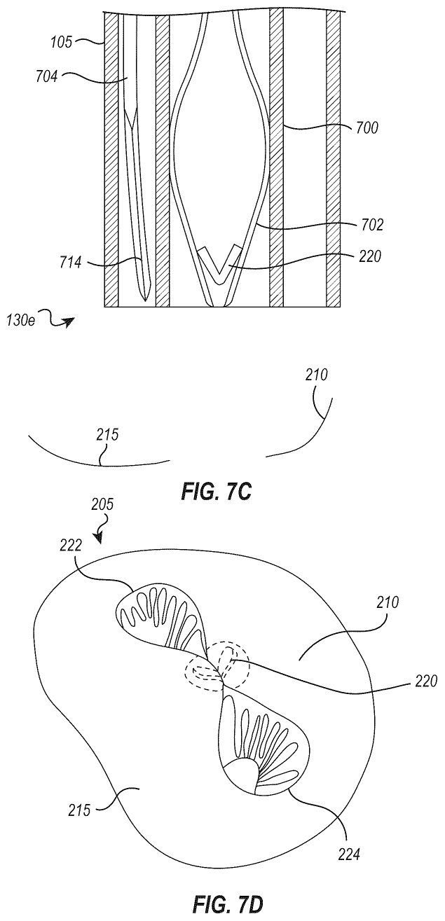

Reference is next made to FIGS. 7A-7D, which illustrate still yet another embodiment of a cutting mechanism for use in detaching an interventional device from one or more leaflets of a mitral valve. Cutting mechanism 130e can include an inner catheter or hypotube 700, a clip grasping structure 702 and an elongated cutter 704. As shown in FIGS. 7A-7C, the distal end 115 of the guide catheter 105 may be positioned above the interventional implant 220. The elongated cutter 704 can be configured to be positioned within the lumen formed between the outer wall of the hypotube 700 and the inner wall of the guide catheter 105 and to selectively extend from the distal end 115 of the guide catheter 105. Cutter 704 can terminate at is distal end in a sharpened point adapted to pierce through leaflet tissue. Cutter 704 can also include one or more sharpened edges 714 (adapted to mechanically cut through the leaflet tissue) that extend along one or both sides of a distal section thereof. The elongated cutter 704 may rotate around the clip grasping structure in an arcuate path, thereby slicing through a portion of leaflet tissue. In at least one embodiment the elongated cutter 704 can be composed of a heat shaped material.

FIGS. 7A-7C further illustrate that hypotube 700 extends through the guide catheter 105 and can be positioned at the distal end 115 of the guide catheter 105. Clip grasping structure 702 may be routable through the hypotube 700 and positioned at a distal end of the hypotube 700. In at least one embodiment, the clip grasping structure 702 can comprise two flexible prongs 706 and 708, each of which preferably includes a sharpened tip adapted to pierce through the tissue of the mitral valve leaflets. The clip grasping structure 702 may comprise an elongated portion 710 and a distal clamping portion 712. As shown in FIGS. 7A and 7B, the distal clamping portion 712 of the clip grasping structure 702 may be extendable distally past the distal end 115 of the guide catheter 105 and the distal end of the hypotube 700. In at least one embodiment, the distal clamping portion 712 can be made of a semi-rigid, resilient metal. Once advanced beyond the distal end and freed from the constraints of hypotube 700, the distal clamping portion 712 can expand laterally as illustrated in FIG. 7A. Once in this configuration, the distal clamping portion 712 of the clip grasping structure 702 may be positioned around the interventional implant 220, and the assembly may then be advanced distally, causing the prongs 706 and 708 of distal clamping portion 712 to puncture through leaflet tissue located to either side of the interventional device 220. Then, as shown in FIG. 7B, hypotube can be advanced distally from the distal end 115 of the guide catheter 105 relative to the clip grasping structure 702, such that the hypotube 700 encloses a proximal portion of the distal clamping portion 712 of the clip grasping structure 702 within the hypotube 700, which also draws the prongs 706 and 708 closer together, thereby causing the distal clamping portion 712 of the clip grasping structure 702 to secure an interventional implant 220. The elongated cutter 704 may then be advanced relative to the guide catheter 105, hypotube 700 and clip grasping structure 702 to extend beyond the distal end 115 of the guide catheter 105 and engage with and cut the leaflet tissue. More specifically, the distal end of cutter 704 may be advanced through the first orifice 222 or the second orifice 224, and then the system can be rotated about its central axis, causing cutter 704 to travel in an arcuate path around the interventional device 220, slicing through the leaflet tissue located adjacent to the interventional device 220 as it travels from one orifice to the other. Of course, the cutter 704 could also be advanced through the leaflet tissue and then the system could be rotated back and forth until the entire cut from one orifice to the other orifice is complete. In addition, with this embodiment it is possible to cut through the tissue of both leaves of the mitral valve, thereby completely separating the interventional device 220 from the mitral valve. In that case, as shown in FIG. 7C, the hypotube 700 and distal clamping portion 712 of the clip grasping structure 702 can be retracted in a proximal direction relative to the guide catheter and into the distal end 115 of the guide catheter 105, thereby securing interventional implant 220. FIG. 7D shows the cut portion 610 of the anterior mitral leaflet 215, however the present invention is not limited to the positioning or shape of cut.

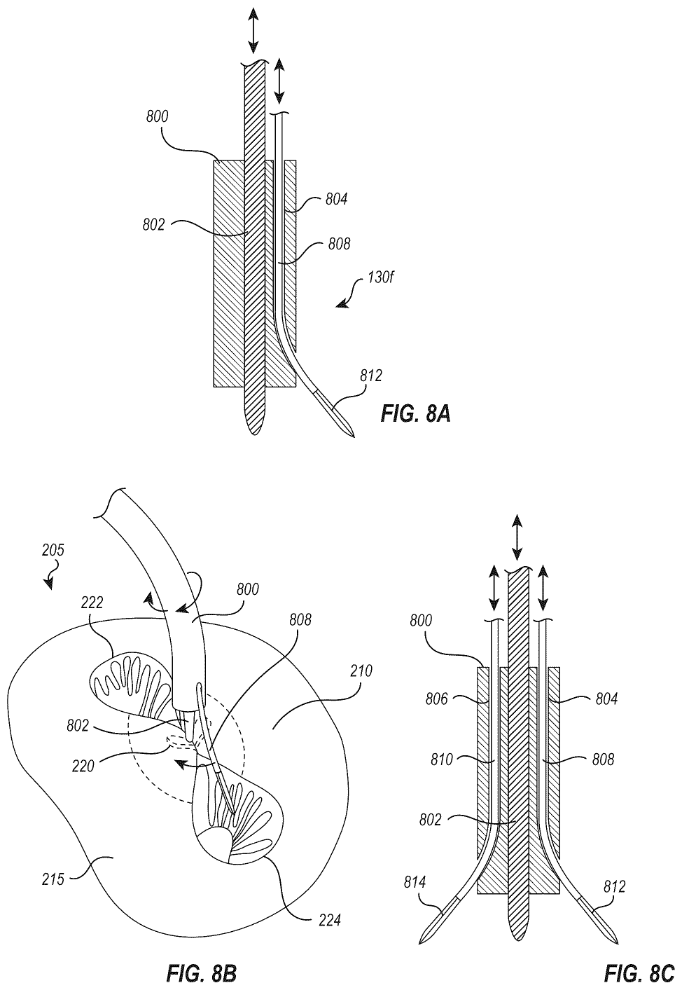

Finally, reference is next made to FIGS. 8A-8C, which illustrate still yet another embodiment of a cutting mechanism for use in detaching an interventional device from one or more leaflets of a mitral valve. In this embodiment, cutting mechanism 130f can include an inner catheter or hypotube 800 and a flexible stabilizing rod or dilator 802. As shown, stabilizing rod 802 extends through the inner lumen of hypotube 800 and can be selectively advanced and/or retracted relative to hypotube 800. The guide catheter is not shown in FIGS. 8A and 8C, but it should be understood that cutting mechanism 130f is intended for use within a suitable guide catheter to position the distal end of cutting mechanism 130f above the interventional device 220. Once properly positioned, stabilizing rod 802 can be advanced relative to the guide catheter and hypotube 800 so as to extend beyond the distal end of hypotube 800 by a predetermined distance. With the stabilizing rod 802 advanced, the entire system can be advanced distally, until the distal end of stabilizing rod 802 engages the fold of leaflet tissue located opposite the interventional clip 220, which fold is created when the posterior and anterior leaflets 210 and 215 are brought and held together by interventional device 220.

As shown in FIGS. 8A and 8C, hypotube 800 can include one or more lumens formed in the wall of hypotube 800, such as lumen 804 shown in FIG. 8A and lumens 804 and 806 shown in FIG. 8B. As illustrated, each lumen 804 and 806, extends from the proximal end of hypotube 800 and then exits at an angle laterally outwardly from the side wall of hypotube 800 near the distal end of hypotube 800. Positioned within each lumen 804 and 806 is an elongated cutter 808 and 810. Each cutter 808 and 810 preferably terminates in a cutting blade portion 812 and 184 at its distal end, and can be selectively advanced and retracted relative to hypotube 800. As shown in FIGS. 8A and 8C, when cutters 808 and 810 are advanced to extend beyond hypotube 800, the distal ends of cutter 808 and 810 splay radially outwardly at an angle relative to the central axis of hypotube 800.

In the case of the embodiment shown in FIG. 8A, once the distal end of the stabilizing rod 802 is firmly engaged against the leaflet fold opposite the interventional device 220 as described above, then cutter 808 can be advanced to extend out and beyond the end of hypotube 800. In some instances, the advancement of cutter 808 will also extend through one of the orifices 210 or 215, but it may also be necessary to further advance hypotube 800 relative to stabilizing rod 802 until cutter 808 extends into orifice 222 or 224. At that point, hypotube 800 alone or hypotube 800 and the guide catheter 105 together can be rotated around stabilizing rod 802, thereby causing the cutting blade 812 of cutter 808 to slice through the leaflet tissue in an arcuate path around interventional device 220 as schematically illustrated in FIG. 8B. With a single cutter 808, a rotation of 180 degrees should be sufficient to detach one leaflet from the interventional device 220, and a rotation of 360 degrees should be sufficient to detach both leaflets from the interventional device. As will be appreciated, the embodiment shown in FIG. 8C operates in a substantially similar manner, except that each cutter 808 and 810 can simultaneously be inserted through both orifices, and cutting of both leaflets can be completed with a single rotation of 180 degrees.

In describing the various embodiments above, the description may at times have explicitly discussed one particular mitral valve leaflet, such as anterior leaflet 215. It should be understood and appreciated, however, that the invention is not intended to be limited to either specific leaflet, but instead can be used to cut either anterior leaflet 215, posterior leaflet 210, or both.

It should also be understood that the order of manipulation of components of the various embodiments as described above are provided as representative examples only, and changes in the order of manipulation that may be readily understood by those skilled in the art are intended to be encompassed within the scope of this disclosure.

Further still, in addition to the embodiments described above, it should also be understood that individual components from one embodiment could also be combined with and/or substituted for a comparable component described in a different embodiment. For example, the stabilizing rod 802 disclosed in relation to the embodiment shown in FIGS. 8A-8C could also be incorporated into one or more of the other embodiments, such as, for example, the embodiment illustrated in FIGS. 3A-3D. Similarly, the cutting blades and structure shown in FIGS. 8A and 8C could be substituted in place of the cutting blade shown in FIGS. 7A-7C, or vice versa.

Similarly, while many of the embodiments discussed above contemplate mechanical cutting of leaflet tissue by means of sharpened edges of a cutting element, it should be further understood that such embodiments could also be adapted to include suitable electrical connections between the cutting element and a source of electrosurgical energy so that such cutting elements may accomplish cutting of tissue by mechanical cutting, by the application of electrosurgical energy to surrounding tissue through the cutting element, or by a combination of both.

Also, with any or all of the foregoing embodiments, one or more components of the leaflet cutting system can also include one or more radiopaque and/or echogenic markers to aid in the visualization of such components during a procedure. For example, one or more radiopaque and/or echogenic markers can be provided on the distal end 115 and/or the steerable portion 117 of the guide catheter 105. Similarly, one or more radiopaque and/or echogenic markers can also be provided on various components of the different embodiments of the cutting mechanisms described above, including, but not limited to such markings being provided on the distal ends of the inner catheter, hypotube, clip grasping structures, cutting blades, stabilizing rods, etc.

One skilled in the art will appreciate that the present invention is not limited to use within the mitral valve. The cardiac valve could also be the tricuspid aortic, pulmonic valve, etc. More generally, the embodiments described herein may be applied in other implementations involving removal of a previously implanted or deployed device from tissue. Further, although figures show the guide catheter 105 extending through the interatrial septum 200, the present invention is not limited to use via a transseptal approach. Any suitable delivery approach may be used, including transfemoral, radial, transjugular, or transapical.

Following are some further example embodiments of the invention. These are presented only by way of example and are not intended to limit the scope of the invention in any way.

Embodiment 1. A system for cutting leaflet tissue at a cardiac valve, comprising a guide catheter having a proximal end and a distal end, wherein the distal end of the guide catheter is steerable to a position above a cardiac valve, a handle coupled to the proximal end of the guide catheter, the handle comprising at least one control configured to steer the guide catheter to the position above the cardiac valve, and a cutting mechanism routable through the guide catheter and able to be positioned at the distal end of the guide catheter, the cutting mechanism configured to cut a portion of leaflet tissue of the cardiac valve.

Embodiment 2. The system of embodiment 1, wherein the at least one control is further configured to provide selective actuation of the cutting mechanism.

Embodiment 3. The system in any of embodiments 1 to 2, wherein the guide catheter is introduced transseptally.

Embodiment 4. The system in any of embodiments 1 to 3, further comprising an indeflator attached to the guide catheter, the indeflator configured to hold the leaflet in place by applying negative pressure.

Embodiment 5. The system in any of embodiments 1 to 4, wherein the guide catheter is U-shaped.

Embodiment 6. The system in any of embodiments 1 to 5, wherein: the cutting mechanism comprises two blades joined at a pivot point, wherein the two blades each comprise a cutting edge disposed on an outside surface of each of the two blades and the two blades are oriented and shaped such that they cut the portion of leaflet tissue of the cardiac valve in a predefined arc; and the cutting mechanism is configured to extend from the distal end of the guide catheter.

Embodiment 7. The system in any of embodiments 1 to 6, wherein: the cutting mechanism comprises a hypotube with a sharped end that terminates in a point, wherein the sharpened end is configured to cut the portion of leaflet tissue of the cardiac valve in a predefined arc; and the cutting mechanism is configured to extend from the distal end of the guide catheter.

Embodiment 8. The system in any of embodiments 1 to 7, wherein: the cutting mechanism comprises a first and a second delivery catheter, each comprising a proximal end and a distal end; each of the first and second delivery catheters comprises a rotatable paddle joined to the delivery catheter by a joint at the distal end of the delivery catheter; each of the first and second delivery catheters comprises a gripping mechanism rotatable around the joint and positioned between the first delivery catheter and the rotatable paddle of the first delivery catheter, and the second delivery catheter and the rotatable paddle of the second delivery catheter; and a wire extending from the joint of the first delivery catheter to the joint of the second delivery catheter.

Embodiment 9. The system in any embodiments 1 to 8, wherein: the first delivery catheter is configured to extend from the distal end of the guide catheter thereby extending the rotatable paddle of the first delivery catheter into a first orifice of the cardiac valve, the gripping mechanism of the first delivery catheter and the rotatable paddle of the first delivery catheter are configured to secure leaflet tissue therebetween, the second delivery catheter is configured to extend from the distal end of the guide catheter thereby extending the rotatable paddle of the second delivery catheter into a second orifice of the cardiac valve, and the gripping mechanism of the second delivery catheter and the rotatable paddle of the second delivery catheter are configured to secure leaflet tissue therebetween.

Embodiment 10. The system in any of embodiments 1 to 9, wherein the gripping mechanism of each of the first and second guide catheters secures the leaflet tissue on an atrial side, and the rotatable paddle of each of the first and second guide catheters secures the leaflet tissue on a ventricular side.

Embodiment 11. The system in any of embodiments 1 to 10, wherein: the wire is configured to rotate around a hub within the joint of each of the first and second delivery catheters; and the wire configured to selectively provide radio frequency current energy to the portion of leaflet tissue of the cardiac valve, thereby cutting the portion of leaflet tissue when rotated around the hub within the joint of each of the first and second delivery catheters.

Embodiment 12. The system in any of embodiments 1 to 11, further comprising a clip grasping structure comprising an elongated portion and a distal clamping portion; wherein: the distal clamping portion of the clip grasping structure is extendable distally past the distal end of the guide catheter and configured to secure an interventional implant that approximates adjacent leaflets of the cardiac valve; and the distal clamping portion of the clip grasping structure is retractable proximally into the distal end of the guide catheter with the secured interventional implant.

Embodiment 13. The system in any of embodiments 1 to 12, wherein the distal clamping portion of the clip grasping structure comprises two prongs.

Embodiment 14. The system in any of embodiments 1 to 13, wherein: the cutting mechanism comprises a cutting wire comprising an elongated portion that terminates in a distal loop portion, the cutting wire extends from the handle to the distal end of the guide catheter, and the distal clamping portion of the clip grasping structure extends through the distal loop portion of the cutting wire when extending distally past the distal end of the guide catheter and retracting proximally into the distal end of the guide catheter with the secured interventional implant.

Embodiment 15. The system in any of embodiments 1 to 14, wherein: the cutting wire is configured to selectively provide radio frequency current energy, and the distal loop portion of the cutting wire is configured to cut the portion of leaflet tissue when the distal clamping portion of the clip grasping structure retracts proximally into the distal end of the guide catheter with the secured interventional implant.

Embodiment 16. The system in any of embodiments 1 to 15, wherein the cutting wire is configured to detach the interventional implant from the surrounding leaflet tissue.

Embodiment 17. The system in any of embodiments 1 to 16, further comprising a hypotube routable through the guide catheter and able to be positioned at the distal end of the guide catheter, wherein the clip grasping structure is further routable through the hypotube and able to be positioned at a distal end of the hypotube.

Embodiment 18. The system in any of embodiments 1 to 17, wherein the hypotube is configured to extend from the distal end of the guide catheter when the distal clamping portion of the clip grasping structure is extended distally past the distal end of the guide catheter such that the hypotube encloses a proximal portion of the distal clamping portion of the clip grasping structure within the hypotube, thereby causing the distal clamping portion of the clip grasping structure to secure an interventional implant.

Embodiment 19. The system in any of embodiments 1 to 18, wherein the cutting mechanism comprises an elongated cutter configured to extend from the distal end of the guide catheter and rotate around a horizontal arc, thereby cutting the portion of leaflet tissue.

Embodiment 20. The system in any of embodiments 1 to 19, wherein the elongated cutter is configured to selectively provide radio frequency current energy to the portion of leaflet tissue of the cardiac valve, thereby cutting the portion of leaflet tissue when rotated around the horizontal arc.

Embodiment 21. A method of cutting leaflet tissue at a cardiac valve within a body, comprising positioning a guide catheter, having a proximal and a distal end such that the distal end of the guide catheter is positioned at a cardiac valve, routing a cutting mechanism through the guide catheter such that the cutting mechanism extends to the distal end of the guide catheter, wherein the cardiac valve is associated with an interventional implant that approximates adjacent leaflets of the cardiac valve, and a cutting mechanism extends from the guide catheter; and actuating the cutting mechanism to cut at a portion of least one leaflet of the approximated adjacent leaflet.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. A system for severing leaflet tissue at a cardiac valve, comprising:
a guide catheter having a proximal end and a distal end;
a handle coupled to the proximal end of the guide catheter;
a first cutter configured to cut leaflet tissue, the first cutter being routable through the guide catheter and postitionable at the distal end of the guide catheter; and
a stabilizer configured to engage leaflet tissue.

2. The system of claim 1, further comprising a hypotube having a distal end and a proximal end, the hypotube having a first lumen formed therethrough, wherein the first cutter is disposed in the first lumen of the hypotube.

3. The system of claim 2, wherein the first cutter includes a blade.

4. The system of claim 2, wherein the first lumen terminates at a first opening formed in a side wall of the hypotube and a first cutter is configured to extend through the first opening.

5. The system of claim 4, wherein the first cutter extends radially away from the hypotube when extending through the first opening.

6. The system of claim 2, wherein the hypotube is configured to rotate around the longitudinal axis of the stabilizer.

7. The system of claim 2 wherein the first cutter is configured to selectively extend beyond the distal end of the hypotube.

8. The system of claim 1, wherein movement of the first cutter is independent of movement of the stabilizer.

9. The system of claim 1, further comprising a second cutter disposed within a second lumen of the hypotube.

10. The system of claim 9, wherein the second lumen terminates at a second opening formed in the side wall of the hypotube and the second cutter is configured to extend through the second opening.

11. A system for severing leaflet tissue at a cardiac valve comprising
a steerable guide catheter having a proximal end and a distal end;
a hypotube routable through the guide catheter;
a dilator disposed in a lumen of the hypotube, the dilator being configured to selectively extend from the distal end of the hypotube;
at least one cutter having a distal end that can be positioned to extend through the hypotube and project radially outwardly therefrom; and
a handle coupled to the guide catheter and configured to rotate the hypotube around the dilator.

12. The system of claim 11, wherein:
the hypotube has at last one opening formed though a side wall of the hypotube, the opening being proximal the distal end of the hypotube; and
at least one cutter extending outwardly through the first opening in the side wall of the hypotube.

13. The system of claim 11, wherein a first cutter of the at least one cutter includes a sharpened edge.

14. The system of claim 11, wherein each of the at least one cutter is configured to be selectively positioned to extend distally beyond the dilator.

15. The system of claim 11, wherein each of the at least one cutter is configured to be selectively positioned to extend radially away from the distal end of the hypotube.

16. The system of claim 11, wherein each of the at least one cutter includes a blade, each of the at least one cutter is configured to be selectively positioned to extend through one respective opening in the side wall of the hypotube.

17. A method comprising:
positioning a distal end of a guide catheter proximate to a cardiac valve;
advancing a cutting assembly distally beyond the distal end of the guide catheter, wherein the cutting assembly includes:
a hypotube having a distal end, a side wall, and a first opening formed in the side wall thereof;
a stabilizing member at least partially positioned within the hypotube; and
a first cutter having a distal end and being at least partially positioned within the hypotube;
advancing the distal end of the stabilizer out of the distal end of the hypotube to engage a fold of the leaflet tissue adjacent to an implant;
advancing the distal end of the first cutter through the first opening and radially away from the side wall; and
severing the leaflet tissue.

18. The method of claim 17, wherein severing the leaflet tissue includes rotating the hypotube relative to the stabilizing member.

19. The method of claim 17, further comprising selectively extending a second cutter through the second opening in the side wall of the hypotube and beyond the distal end of the hypotube.

20. The method of claim 17, wherein the implant is fixation device.
